(19) United States
(12) Patent Application Publication
(10) Pub. No.: US 2020/0176099 A1
(43) Pub. Date: Jun. 4, 2020

(54) METHOD AND APPARATUS FOR ESTABLISHING A BODY REGION STATE

(71) Applicant: Henkel AG & Co. KGaA, Duesseldorf (DE)

(72) Inventors: THOMAS WELSS, Duesseldorf (DE); HANS GEORG KNUEBEL, Duesseldorf (DE)

(73) Assignee: Henkel AG & Co. KGaA, Duesseldorf (DE)

(21) Appl. No.: 16/616,036

(22) PCT Filed: Jun. 5, 2018
(86) PCT No.: PCT/EP2018/064718

§ 371 Date: Nov. 22, 2019

(30) Foreign Application Priority Data
Jun. 12, 2017 (DE) 10 2017 209 860.0

(51) Int. Cl. G16H 20/00; A61B 5/00; G16H 50/30; G16H 30/40; G06Q 30/06; G06T 7/90

(52) U.S. Cl. CPC G16H 20/00; G06T 2207/30036; A61B 5/444; A61B 5/4547; A61B 5/448; A61B 5/0075; A61B 5/0077; A61B 5/6898; A61B 5/449; G16H 50/30; G16H 30/40; G06Q 30/0631; G06T 7/90; G06T 2207/30088; A61B 5/443

(57) ABSTRACT

A method for determining a body area condition is provided. The method may includes determining at least one body condition parameter and determining the body area condition using the at least one body condition parameter. The determining of the body condition parameter includes recording, during a sequential exposing of a body area of a user to light of different colors by a display of a portable data processing device, a plurality of images of at least a part of the light which has interacted with the body area, determining a spectral characteristic of the body area by the plurality of images, comparing the spectral characteristic with calibration spectral characteristics obtained for a plurality of calibration body areas, and determining the body condition parameter including a result of the comparison.

### CROSS-REFERENCE TO RELATED APPLICATION

This application is a U.S. National-Stage entry under 35 U.S.C. § 371 based on International Application No. PCT/EP2018/064718, filed Jun. 5, 2018, which was published under PCT Article 21(2) and which claims priority to German Application No. 10 2017 209 860.0, filed Jun. 12, 2017, which are all hereby incorporated in their entirety by reference.

### TECHNICAL FIELD

Various embodiments of the present disclosure generally relate to a method and device for determining a body area condition and to a method for determining a body treatment product.

### BACKGROUND

In many domains, a selection of a suitable product may depend on a current condition of a body area. The collective term “body area” is used herein for skin (including lips), hair, teeth and nails.

For example, different products may be suitable, depending on whether, for example, a skin is dry or greasy, hair is untreated or dyed/bleached and/or teeth are heavily or lightly plaque-coated.

For example, healthy and young-looking skin (normal skin) may have a small-pored, taut appearance. Normal skin may feel supple and neither too dry nor too oily.

In contrast, dry skin may appear flaky, rough, and/or sensitive to external influences. Older people may often be affected by dry skin.

Dry skin may be due, for example, to a disruption of a barrier function of the skin due to an inappropriate amount of skin lipids and/or due to a wrong composition of the skin lipids.

Various special face and/or body cleaning and body treatment products may be available for the special care of dry skin.

An excess of skin lipids may be present on the skin surface when there is oily skin, which may occur, for example, in particular in the years after puberty, for example, especially in the face. This may promote the growth of microorganisms, which may lead to unclean skin. The oily skin may appear coarse-pored and shiny.

Various special cleaning and care products, especially for the face, may also be available for the special care of oily skin.

Similarly, depending on a degree of plaque load on the user's teeth, a recommendation to brush the teeth more frequently or to use mouthwash may be sufficient (optionally suggesting which mouthwash the user could use or which toothpaste or toothbrush the user could use) or, should it be necessary, recommending a visit to a dentist.

As a further example, colored hair may have color inhomogeneities. Possible measures for eliminating the color inhomogeneities, for example, towards a homogeneous hair color, may depend on a hair condition of the user, for example, on the hue (possibly, for example, of a type of coloring agent used to dye the hair) at a location of the hair style on which the color inhomogeneities occur, etc. Suitable care instructions (or products) may have, for example, re-dyeing or tinting the hair or partial region of the hair, over-coloring all the hair in a different color, and/or cutting the hair. Furthermore, it may be desirable, for example, when using a hair supplement part, to select a color of the part in such a way that it resembles the hair to be supplemented in any type of lighting as far as possible.

Without expert advice, for example, dermatological or cosmetic advice or advice from a dentist or hairdresser, it may be difficult for a user to determine his individual body area condition and the cosmetics and/or care instructions suitable for his body area condition.

If the user still consumes cosmetic products and/or uses care instructions, it may be virtually impossible to reconstruct a treatment success, because the user, for example, at home, lacks opportunities to assess a treatment outcome in a standardized and objective manner.

As a result, it may be difficult for the consumer to assess the individual effectiveness of a cosmetic and/or a care instruction, which may result in a motivation to perform a corresponding cosmetic treatment, for example, in the longer term, being impaired. This may be the case even if a cosmetic product were suitable for achieving an objectively detectable desired effect.

In many areas of daily life, there has been a trend for some time towards personalized programs that may respond in a targeted manner to individual requirements and needs, for example, in a nutrition or health area.

Spectroscopic examination methods may be applied to determine body area parameters, for example, in a laboratory, a hair salon, at the dentist or in a beauty salon. However, it may be necessary to use a spectrometer which may be expensive, bulky/unwieldy and/or complicated to operate.

### SUMMARY

In various embodiments, a device and method for determining a body area condition of a user may be provided that uses, instead of a spectrometer, a portable data processing device (for example, a smartphone, a tablet, or an iPad) with specialized software, such as an app to determine a spectral characteristic of a body area.

In various embodiments, a device for determining a body area condition of a user may be provided. The device includes a portable data processing device that may be applied to a (live) user, for example, the user at home. In various embodiments (for example, when a smartphone is used as the sensor), the portable data processing device may have a size that makes it possible to easily place the device in a pocket or trouser pocket, for example, with an area of less than about 36 cm² and a thickness of less than about 2 cm.

In various embodiments, the device for determining the body area condition may be configured, for example, by the software/app, to take a plurality of recordings of the body area, wherein the body area is illuminated with a different color during at least one recording of the plurality of recordings. In this case, the illumination in various embodiments may be effected by employing a display of the portable data processing device, and the recording may take place by employing a camera having its inlet aperture on the side of the portable data processing device on which the display is located. It may thus be achieved that light which is radiated from the display in the direction of the body area, after interaction with the body area, may be radiated in the direction of the camera and recorded by the camera. In this case, an amount of light of the differently colored light, after interacting with the body area, may depend on an absorption characteristic of the body area. For example, for at least one, for example, several or all colors, a part of the light may be absorbed by the body area and thus not be re-radiated to the camera. The different colors may be white in various embodiments (that is, a broad spectral range), for example, from the blue spectral range (from about 380 nm) to the red spectral range (up to about 780 nm) or possibly even into a near-infrared wavelength range (for example, up to about 1 μm), having red, green and blue. In various embodiments, those colors provided by individual monochrome sub-pixel light elements of the display may be used as the various colors with which the body area is illuminated. In various embodiments, one or more of the different colors with which the body area is illuminated may be generated by superimposing the light of a plurality of the single monochrome sub-pixel light elements of the display.

A method substantially known to the person skilled in the art may be used, which method is used for recording the spectral characteristic, for example, in an app developed by the Fraunhofer Institute for Factory Operation and Automation (HawkSpex® mobile).

Based on the absorption determined in the recording (which contains an image of at least the body area and therefore sometimes referred to herein as simply “image”), in various embodiments, at least one body area condition parameter may be determined, for example, a content of a substance present in the body area of the user and causing the absorption. In various embodiments, a body area condition of the user may be determined using the at least one body area condition parameter. In various embodiments, contents of a plurality of substances in the skin of the user may be determined as a plurality of body area condition parameters that may be taken in isolation and/or related to one another to determine a user's body area condition therefrom, and/or colors (for example, in an L*a*b* representation) of hair and/or teeth (also, for example, stained plaque on the teeth) may be determined.

A device and method for determining a body area condition may be provided in various embodiments. At least one body area condition parameter may be determined in various embodiments for this purpose. The device, method, and/or at least one body area condition determined by the device or method may be utilized to determine the needs of body areas (for example, skin, hair, or teeth) of the user.

In various embodiments, a user is to be enabled to find targeted cosmetic products and/or to obtain care suggestions that may be tailored to the individual needs of the user's body area.

In various embodiments, skin elasticity and/or skin moisture may be determined in an anti-aging area (wherein a collagen content and/or a water content of the skin may be relevant for this), so that care for specific skin types (for example, dry skin, oily skin, elastic skin, inelastic skin) may be determined and recommended, and/or, for example, because an oxygen saturation of the skin, which is able to be determined in the presence of dark circles (for example, the hemoglobin in the blood in the skin), may be reduced.

The at least one body area condition parameter includes an oxygen saturation, a water content, a lipid content, a protein content, in particular a collagen content, a content of a compound from the group of advanced glycation end products (AGEs), a content of fine dust, a pollen content, a mite content, a cysteic acid content, a melanin content, a silicone content, a thiol content, a content of Bunte salt, a content of disulfide with thioglycolate unit, a content of antioxidants or the like.

In various embodiments, the at least one body area condition parameter includes a measure of a proportionate coverage (also referred to as degree of coverage) of teeth having plaque, wherein prior to determining the degree of coverage of the teeth having plaque, the plaque may have been stained by employing a suitable dye that selectively stains the plaque and not the teeth).

In various embodiments, the at least one body area condition parameter includes a hair color, for example, a hair color parameterized in a L*a*b* color space.

In various embodiments, the at least one body area condition parameter includes a skin tone, for example, a skin tone parameterized in an L*a*b* color space. Certain influences (UV light irradiation, irritation by products, vasodilation by drugs, actinic keratosis, etc.) increases the redness of the skin (for example, locally), for example, in the form of sunburn, erythema or reddish-brown flaky skin) and the a-value increases. Increasing browning of the skin (for example, locally due to pigment spots/age spots, black skin cancer) is described by an increasing b*value and a decreasing L*value.

In various embodiments, the at least one body area condition parameter includes a wrinkle quality that is determined via the depth and/or extent and/or number of wrinkles per determined area.

The at least one body area condition parameter may be utilized individually in various embodiments to determine a body area condition. In various embodiments, a plurality of the determined body area condition parameters may be utilized in combination to determine a, for example, a single, body area condition.

For determining the at least one body area condition parameter and/or for determining the body area condition, in various embodiments, a software, for example, an app, which is utilized on the portable data processing device, may be installed. The software/app may measure, using the display and the camera, relevant parameters of the skin, hair and/or teeth of the user, for example, a water content (that is, a moisture), a lipid amount, a collagen amount, an oxygen saturation of the skin, which enables conclusions about the microcirculation of the skin, which constitutes an important factor for a skin color, a degree of coverage of the teeth with coating, in particular plaque, a uniform hair, skin and/or tooth color, a content of fine dust on the skin, which enables conclusions about the degree of soiling of the skin, a content of (allergy-causing) pollen and/or mites on skin and/or hair, which enables conclusions about the probability and/or intensity of allergic reaction on the part of a user, a skin color, whose (local) deviation from the norm enables conclusions about skin changes, a cysteic acid content of hair, which enables conclusions about the degree of oxidative damage to hair, a content of sulfur species such as thiol, which enables conclusions about the degree of oxidative damage to hair, and/or another relevant and/or interesting parameter.

In various embodiments, by employing the portable data processing device, for example, by employing the software/app, the determined parameters may be represented or communicated as values (for example, with arbitrary units), as a verbal message, as a graphic representation, or the like.

In various embodiments, at least one body treatment product may be recommended to the user based on the determined body area condition. The body treatment product may be suitable for maintaining or improving the body area condition of the user (for example, increasing the oxygen and/or moisture content of the skin).

In various embodiments, the body treatment product may comprise a skin treatment product, in particular a care and/or cleaning product, such as a cream, a lotion, an ointment, an oil, an emulsion, a gel, a soap, a mask, an ampoule with an additional care product, a facial toner, a serum, a spray or the like.

In various embodiments, the body treatment product may comprise a hair treatment product such as a coloring agent, a blonding agent, a styling agent, a conditioner, a cleaning agent, or the like.

In various embodiments, the body treatment product may comprise an oral hygiene product such as a toothpaste, a mouthwash, a gel or finish for application to the teeth and/or the gums, an oral spray or a chewing gum.

In various embodiments, the body treatment product may comprise a food and/or nutritional supplement containing certain minerals, vitamins, and/or antioxidants.

Care instructions comprise, in particular, tips and recommendations for the cleaning and treatment of skin, hair and/or teeth with the aim of maintaining the natural protective mechanisms and the health of the skin, hair and/or teeth and improving general well-being.

In various embodiments, recommendations for individually tailored body treatment products and/or individual care instructions for the user may be derived based on the determined body area condition parameters and/or on the basis of the determined body area condition. The product recommendations and/or care instructions may be provided, for example, by employing software, for example, an app.

In various embodiments, the user may be encouraged to use body treatment products that are manufactured individually for the user and may initiate an ordering process, preferably by accessing a manufacturer's website of individual body treatment products.

More and more customers desire a product that is individually tailored to their needs. This may be a product manufactured especially for the customer or a so-called “mass customized” product. In the case of a “mass customized” product, an individualization may be achieved by varying a few characteristics of a product that are decisive from the customer's point of view. Preferably, these “mass customized” products are based on the concept of modularization, that is, the product may be assembled individually from various modules/building blocks.

Often, there are many dependencies between the many different features/ingredients of a product, which may be expressed as “commandments” or “prohibitions.” In order to obtain a clear product definition, it may be advantageous for the ordering process to proceed with the aid of a product configurator. This configurator helps the customer to select the characteristics/ingredients and draws attention to the permitted/inadmissible combinations of features, wherein the latter then cannot be selected.

With body treatment products, the relevant product characteristics comprise, in particular, the chemical ingredients of the agent, the physical properties of the agent and the manner of preparation of the agent. With the help of a product configurator, for example, the selection of chemically and/or physically incompatible ingredients or the selection of the ingredients unsuitable for the determined body area condition may be avoided. Conversely, the selection of suitable ingredients for the determined body area condition may be predetermined or suggested by the product configurator.

In various embodiments, the visit to a physician, in particular a dermatologist or dentist, a hairdresser or a beautician may be recommended. In various embodiments, a booking process may be initiated directly via the software/app, which determines the body area condition and/or the product recommendation and/or the care instruction. For this purpose, for example, the contact data of physicians and hair stylists may be stored in the software/app and these may be displayed to the user.

In addition, the selection may be restricted via filters, such as the postal code. In various embodiments, an appointment booking may be made directly via the software/app. Alternatively, the booking of a physician appointment and/or a cosmetic appointment may be made via a separate software/app, such as Treatwell.

In various embodiments, the software/app that determines the body area condition parameter and/or the body area condition may be the same as that which determines the product recommendation and/or the care instructions. In various embodiments, different software programs/apps may be used for a part of the various operations or all of the various operations (determining the at least one body area condition parameter, determining a product recommendation, determining a care instruction).

In various embodiments, a treatment success in a treatment, which may have as a goal a positive effect on the determined body area condition parameter or the determined body area condition, may be monitored. In various embodiments, the software/app may enable control and/or tracking of the results by employing a representation (for example, a graphical representation) of the measurement results over time.

In various embodiments, when determining the body treatment product and care recommendations, further information regarding a general health condition, age, sex, a further body area condition, eating habits, sports habits, and other user behaviors (for example, daily length of stay outdoors/in the sun/in the water, smoking habits, etc.), may be used, for example, by the software/app.

In various embodiments, literature data may be the basis of assessing suitability of a body treatment product and/or a care instruction in caring for skin, hair or teeth having a given body area condition.

In various embodiments, each body area condition of a plurality of body area conditions may be associated with a quality score. A body treatment product and/or a care instruction may be rated as suitable for a body area condition when it is to be expected, for example, on the basis of literature data, test results or empirical values, that with an (for example, regular) application of the body treatment product and/or the care instruction, the body area condition of the user is maintained or changed to a body area condition having a higher quality value.

In various embodiments, an assessment of a suitability of a body treatment product to improve a body area condition may be confirmed or modified by receiving empirical values of further users having the same or a similar body area condition, for example, empirical values with regard to treatment success. In particularly preferred embodiments, the further users have a similar profile in terms of age, sex, eating habits and/or other behaviors. It is thus possible for the user to always receive an optimal recommendation.

In various embodiments, it may be possible to check and track the effectiveness of a treatment in an objective and standardized manner. In various embodiments, the treatment may have the aim to increase a skin hydration and/or to improve a collagen quantity and/or structure, an oxygen saturation of the skin and/or its microcirculation, to reduce a degree of coverage of teeth with coating, in particular plaque, and/or to achieve a uniformity of hair, skin and/or tooth colors.

In various embodiments, an efficacy of a (for example, cosmetic) treatment may be better tracked and a selection of an individually suitable product may thereby be simplified.

In various embodiments, a user's motivation may be increased to perform a treatment in the longer term, for example, by a comparison with other users, for example, by employing information about treatment successes provided by the other users.

In various embodiments, a method for determining a body area condition is provided. The method comprises the steps of determining at least one body condition parameter and determining the body area condition using the at least one body condition parameter. The determining of the at least one body condition parameter includes the steps of during a sequential exposing of a body area of a user to light of different colors by a display of a portable data processing device, recording a plurality of images of at least a part of the light which has interacted with the body area, determining a spectral characteristic of the body area by the plurality of images, comparing the spectral characteristic with calibration spectral characteristics obtained for a plurality of calibration body areas, and determining the at least one body condition parameter including a result of the comparison.

The comparison of the spectral characteristic with calibration spectral characteristics obtained for a plurality of calibration body areas and/or the determination of the at least one body condition parameter including a result of the comparison may be carried out with the aid of an evaluation algorithm.

This may be an artificial system that learns, for example, from the calibration spectral characteristics and may generalize them after completion of the learning phase. That is, the examples are not simply learned by heart, but rather patterns and principles are recognized in the learning data. Different approaches may be followed for this purpose. For example, supervised learning, partially supervised learning, unsupervised learning, empowered learning, and/or active learning may be used, in particular in conjunction with deep learning methods. Supervised learning may be done, for example, by employing an artificial neural network (such as a recurrent neural network) or by employing a support vector machine. Unsupervised learning may also take place, for example, by employing an artificial neural network (for example, an auto-encoder).

In various embodiments, determining the at least one body area condition parameter includes determining a substance content and/or determining a ratio of substance contents.

In various embodiments, the at least one body area condition parameter includes at least one from a group of body area condition parameters, the group having a water content, a lipid content, a protein content, in particular a collagen content, an oxygen saturation, a content of a compound from the group of advanced glycation end products (AGEs), a content of fine dust, a pollen content, a mite content, a cysteic acid content, a melanin content, a silicone content, a thiol content, a content of Bunte salt, a content of disulfide with thioglycolate unit, a content of antioxidants, a skin tone, a tooth color, a polysaccharide content, a degree of coverage of teeth with coating, in particular plaque, and a hair color.

In various embodiments, the at least one body area condition parameter includes a plurality of body area condition parameters.

In various embodiments, exposing the body area includes exposing a plurality of body areas of the user.

In various embodiments, determining the body area condition includes determining a single body area condition including the plurality of body area condition parameters and/or the body area condition parameters for the plurality of body areas.

In various embodiments, the body area includes a skin area, a hair area, and/or a tooth area.

In various embodiments, the portable data processing device includes a smartphone, a tablet or an iPad.

In various embodiments, the method may further have recording at least one dark shot while the body area of the user is not exposed to light from the display of a portable data processing device.

In various embodiments, a method for determining a product for skin treatment is provided. The method includes the steps of determining a body area condition according to various embodiments and determining the product based on the determined body area condition and a database that includes a plurality of body area conditions and a plurality of associated products, wherein each body area condition of the plurality of body area conditions is associated with at least one suitable body treatment product.

In various embodiments, each body area condition may be associated with a quality score, and the body treatment product may be suitable for at least one body area condition of the plurality of body area conditions when an improvement or maintenance of the body area condition quality score is to be expected with the body treatment product based on stored empirical values.

In various embodiments, the method may further have updating the database based with new empirical values from a plurality of (further) users.

In various embodiments, determining the at least one body area condition parameter and/or determining the body area condition includes transmitting the plurality of images and/or the result of the comparison to an external data processing device and receiving the body area condition parameter and/or the body area condition.

In various embodiments, a device for determining a body area condition is provided. The device comprises a portable data processing device having a display for sequentially exposing a body area of a user to light of different colors by the display and a camera for recording a plurality of images of at least a part of the light interacting with the body area, wherein the portable data processing device is configured to determine a spectral characteristic of the body area based on the plurality of recorded images, to determine at least one body area condition parameter based on the spectral characteristic by comparing the spectral characteristic with calibration spectral characteristics obtained for a plurality of calibration body areas, and to determine a body area condition based on the at least one body area condition parameter.

In various embodiments, the portable data processing device may be coupled to an external data processing device by a wireless data transmission, wherein the portable data processing device may be configured to determine the at least one body area condition parameter by initiating the determining, by the external data processing device, of the at least one body area condition parameter, and/or at least one body area condition, and wherein the device may be further configured to receive the at least one determined body area condition parameter and/or the at least one body area condition and to provide this to the user by the portable data processing device.

In various embodiments, the external data processing device may be a processor cloud architecture (also referred to as a “cloud”).

### BRIEF DESCRIPTION OF THE DRAWINGS

The present disclosure will hereinafter be described in conjunction with the following drawing figures, wherein like numerals denote like elements.

In the drawings, like reference characters commonly refer to the same parts throughout the several views, wherein for the sake of clarity, partial omission is made to provide reference numerals to all corresponding parts throughout the figures. Parts of the same or similar type may be provided with a trailing digit to distinguish them in addition to a common reference numeral. The drawings are not necessarily intended to depict scale, but rather the emphasis is on illustrating the principles of the present disclosure. In the following description, various embodiments as contemplated herein are described with reference to the following drawings, in which:

FIG. 1A and FIG. 1B each show a device for determining a body area condition during an execution of a method for determining a body area condition according to various embodiments;

FIG. 2 illustrates body area for applying a method and device for determining a body area condition according to various embodiments;

FIG. 3 illustrates an example application of a method for determining a body condition or a product and/or a care instruction for treating body areas according to various embodiments;

FIG. 4 illustrates an example application of a method for determining a body condition or a product and/or a care instruction for treating body areas according to various embodiments;

FIG. 5 shows a diagram illustrating a method for determining a product and/or a care instruction for treating body areas according to various embodiments;

FIG. 6 shows a flow chart of a method for determining a body area condition according to various embodiments; and

FIG. 7 shows a flowchart of a method for determining a product and/or a care instruction for treating a body area according to various embodiments.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

may

The term “exemplary” is used herein to mean “serving as an example, exemplar or illustration.” All embodiments or designs described herein as “exemplary” are not necessarily to be interpreted as preferred or advantageous over other embodiments or designs.

In various embodiments, the device for determining a body area condition includes a portable data processing device having a display, for example, a smartphone, a tablet, an iPad, or the like. In the following, in particular in a figure description, the term “smartphone” is sometimes used in a simplifying manner for the portable data processing device. It should be understood that, instead of a smartphone, a suitable other portable data processing device (for example, a tablet, an iPad, etc.) may be used.

In various embodiments, the portable data processing device includes a camera for recording at least one image of visible light, for example, in a wavelength range from about 380 nm to about 780 nm. The camera may also be suitable for recording near-infrared light, for example, in a wavelength range of from about 780 nm to about 1 μm, as is the case with many conventional smartphones, for example. In various embodiments, the recorded light includes at least a part of the light that has been radiated from the display of the portable data processing device to the body area and has interacted with the body area.

The portable data processing device includes a processor and a memory in various embodiments.

The portable data processing device may be configured in various embodiments to determine at least one body area condition parameter based on the recorded images. Furthermore, in various embodiments, the portable data processing device may be configured to determine a body area condition based on the at least one body area condition parameter. In various embodiments, the portable data processing device may itself be configured, that is, directly, to determine the at least one body area condition parameter and/or the body area condition.

In various embodiments, the portable data processing device may be configured to indirectly determine the at least one body area condition parameter and/or the body area condition, for example, by providing the recorded images and/or the body area condition parameter to an external data processing device that may be configured to determine the body area condition parameter and/or the body area condition from the provided data, and receive a body area condition parameter and/or a body area condition from the external data processing device. The body area condition parameter and/or the body area condition may be provided to the user after direct or indirect determination, for example, by employing displays, for example, on a screen of the data processing device.

In various embodiments, the light that has interacted with the skin of the user after having been radiated from the light source of the portable data processing device (for example, from the display, optionally, an integrated (white light) illumination of the smartphone, for example, the flash, may be used in different embodiments for the light in one of the different colors) to the skin may have at least one absorption structure. The absorption structure may be caused by at least one substance present in and/or on the skin, for example, one or more skin components, which substance absorbs part of the light radiated to the skin at at least one wavelength characteristic of the substance (also referred to as an absorption wavelength). At least a part of the remaining light reflected from the skin, scattered or, in general terms, radiated to the camera and as light that has interacted with the skin, may be referred to as received light or as recorded light.

In various embodiments, the substance includes water, a lipid (for example, triglyceride, cholesterol ester, phospholipid, cholesterol), a protein (for example) collagen, oxygen (for example, oxygenated and deoxygenated hemoglobin), and/or another skin, hair or tooth component or one such.

In various embodiments, an absorption intensity, that is, a measure of what proportion of the light irradiated to the skin is recorded by the recording device, may depend on a substance-specific absorption coefficient and on an amount of the substance that may be present in the body area. For example, the absorption intensity may be proportional to the amount of the substance.

Absorption and reflection properties of the body area may determine its color. Colors that may appear the same for a viewer may have different spectral profiles. This property is also called metamerism.

Typically, metameric body colors (that is, non-luminous colors) look the same only when illuminated with light of one kind (that is, a spectral distribution), and appear different for a viewer when the metameric colors are illuminated with light of a different kind, that is, another spectral distribution.

In various embodiments, spectral properties of colors of at least one body area may be determined. On the basis of a spectral characteristic determined by the method or device, color information may be associated with each image element as a parameterization in a color space, for example, by employing a convolution of the ascertained spectral characteristic with sensitivity curves representing a spectral sensitivity of the human eye and a parameterization of the obtained color-brightness values in a known color space, for example, in an L*a*b color space (wherein L indicates the brightness of a color, a the green and red part and b the blue and yellow part of the color), in an RGB color space by color components in red, green and blue, in a CMYK color space by color components in cyan, magenta, yellow and black or in any other color space, such as a CIE LUV color space.

According to various embodiments, the color information may be coupled to other data. For example, the color information may also have information about the body part (location of the measured skin, position of the tooth), information about time of measurement, type of measurement, type and values of calculated values and parameters, and other/further data.

According to various embodiments, a color information may be relative, for example, the color information item may constitute a difference to another color information, for example, the difference from a color of a further body area, for example, as described elsewhere, a color difference between different hair areas, hair of the user and a complementary hair part, a tooth and a dental prosthesis, or for example, a color difference between a masking cream (for example, make-up) applied to a skin area and a natural skin color, which may be, for example, at a skin site next to the site with the masking cream.

In various embodiments, for a recommendation of a product in such a case, a tolerable limit for the color difference may be set, and only a body treatment product having a color which differs by a maximum of the tolerable limit from the color of the body area may be considered worthy of recommendation and recommended to the user.

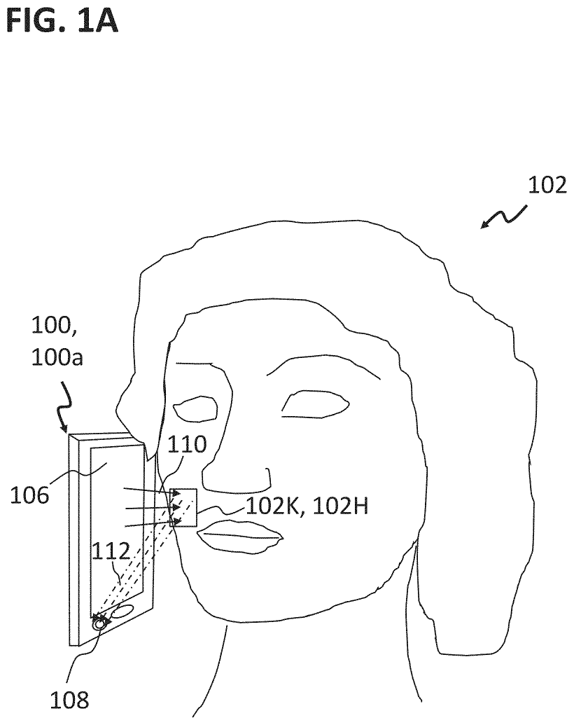

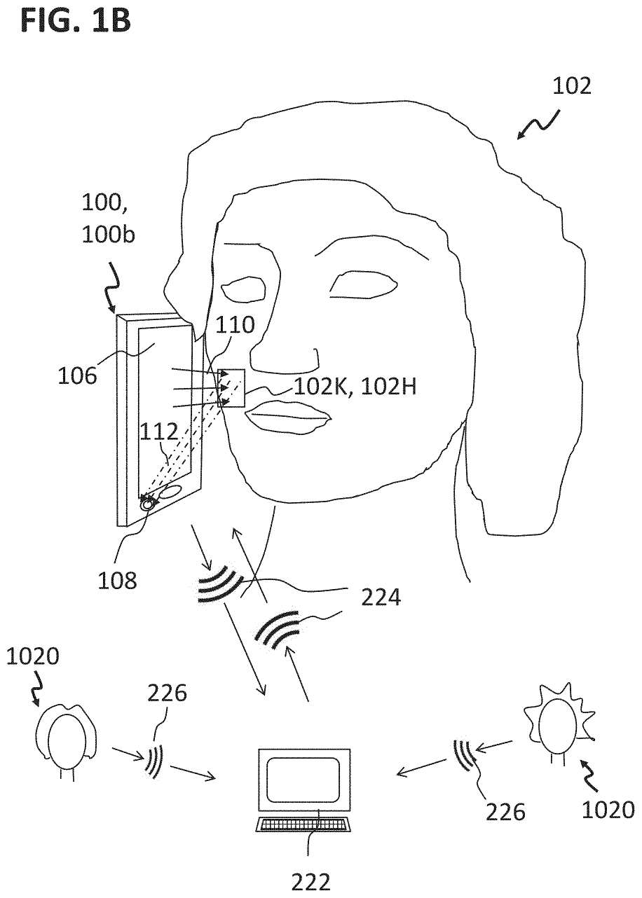

FIG. 1A and FIG. 1B each show a device 100 (100a or 100b) for determining a body area condition of a user 102 according to various embodiments.

The device 100a, 100b for determining a body area condition according to various embodiments may be a portable data processing device 100 (for example, a smart phone, tablet, iPad, or the like) including a light source 106 for exposing/illuminating a body area 102K with light 110 of different colors and a camera 108 for recording a plurality of images of the body area 102K.

The light source 106 can, as illustrated in FIGS. 1A and 1B, be a display 106 of the portable data processing device 100 according to various embodiments. In various embodiments, the light source 106 may further have an (additional) illumination device of the smartphone 100, for example, a lamp or a flash (not illustrated), which may be used in addition to exposing the body area 102K to light of a color (usually white or near white).

The light 110 can, in various embodiments, have visible light, for example, having a wavelength in a range from about 380 nm to about 780 nm, or in one or more subregions within this range, for example, the aforementioned subregions.

In particular, the subregions may comprise from about 600 nm to about 780 nm, from about 490 nm to about 600 nm, from about 380 nm to about 490 nm, or a combination of two subregions.

A wavelength range of from about 600 nm to about 780 nm corresponds to an exposure in the color red. A wavelength range of from about 490 nm to about 600 nm corresponds to an exposure in the color green. A wavelength range of from about 380 nm to about 490 nm corresponds to an exposure in the color blue.

The light 110, in various embodiments, includes near-infrared light in addition to the visible light, for example, having a wavelength in a range of greater than about 780 nm to about 1 μm.

In various embodiments, exposing/illuminating the body area 102K with light 110 includes sequentially exposing body area 102K to a plurality of different colors. For example, the body area 102K may first be exposed to light of a first color by the light source 106. Meanwhile, at least a first image of the body area 102K may be recorded by the camera 108. In various embodiments, multiple images per color (a plurality of first images here) may be recorded and averaged, for example, in the same color. In various embodiments, the body area may be exposed to light of a second color 110 after exposure to the light of the first color. Meanwhile, at least a second image of the body area 102K may be recorded by the camera 108. Further colors may be performed accordingly.

In various embodiments, the first color may be one of those colors that may be generated by employing a single monochrome sub-pixel light element of the display 106.

For example, it is customary to have one red, one green and one blue subpixel in each pixel of the display, in some cases further supplemented by a white subpixel. In various embodiments, the subpixel includes other colors. In various embodiments, one or more of the different colors with which the body area is illuminated may be generated by superimposing the light of a plurality of the single monochrome sub-pixel light elements of the display, for example, by simultaneously emitting light 110 through the red and blue sub-pixels.

A sequential illumination may in particular take place within such a short time interval that the sequential illumination is perceptible by a user as one process.

In various embodiments, the camera 108 may image the body area 102K onto a plurality of photosensitive pixels, which may be divided into a plurality of sub-picture elements provided with different filters. In various embodiments, the filters may be designed in such a way that the respective sub-picture element registers only incident light having a wavelength for which the filter of the sub-picture element is permeable. Usually, filters for red, green and blue may be used. In various embodiments, other combinations of filter wavelength ranges may be used. Frequently, a sensitivity of red sub-picture elements in a smartphone may still easily extend into the near-infrared region, for example, up to about 1 μm.

The device 100 thus has, in various embodiments, the light source 106, which may be configured to generate a plurality of different colors (usually three primary colors, optionally additionally white), and the camera 108, which may be configured to differentiate at the pixel level between light of different wavelength ranges (usually three).

When the body area 102K is illuminated with the light 110 of a predetermined wavelength range, the body area 102K may interact with the light 110 only in this wavelength range, that is, absorb or reflect the light 110 as a function of substances contained in the body area 102K.

In various embodiments, this principle may be used to determine a spectral characteristic of the body area based on the images which were recorded by differently colored filters, while the body area 102K was illuminated with different colors, for example, by a method which is used an app (HawkSpex® mobile) developed for factory operation and automation by the Fraunhofer Institute.

In various embodiments, an image may further be recorded by the camera 108 while the body area 102K is not illuminated by the light device 106. The recording (also referred to as dark shot) may be used to remove a backlight in an evaluation of the recordings, for example, by subtracting the dark shot from a respective illuminated recording.

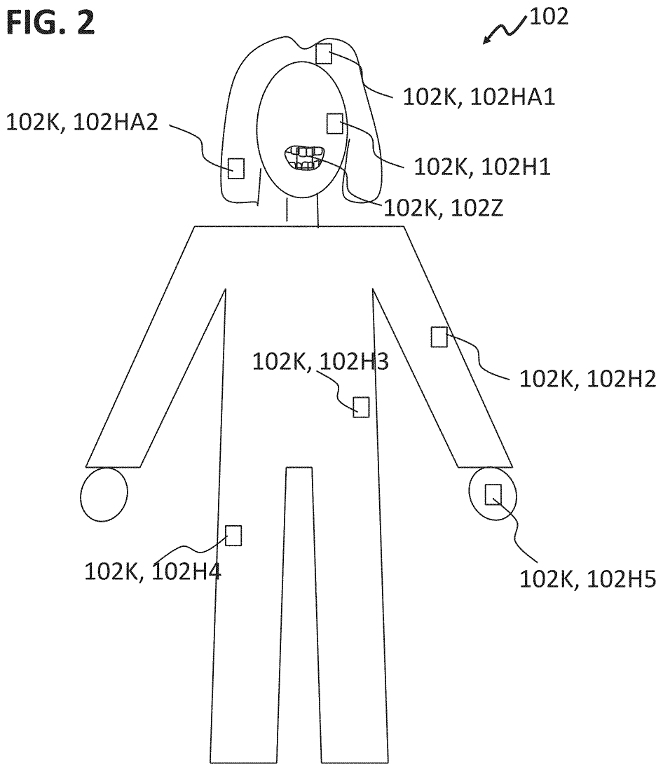

The body area for which a body area condition is determined can, in various embodiments, as shown in FIG. 1A and FIG. 1B as a skin area, be part of a user's facial skin. As shown in FIG. 2, which shows body areas 102K for applying a method and a device for determining a body area condition according to various embodiments, may alternatively or additionally be another body area 102K, 102H222 for example, on a face (area 102H1), an arm (skin area 102H2), a leg (skin area 102H4), a trunk (area 102H3), a hand (area 102H5), various hair areas (102HA1 or 102HA2), and/or teeth 102Z of a user for determining a body area condition of that body area 102K.

Different body areas 102K may have different body area conditions, for example, the skin on the face of the user 102 may be dry, normal on the rest of the body. In a case of different body area conditions in different body areas 102K, the different body areas 102K may require a different treatment. However, different body areas 102K may also have the same or similar body area conditions.

In various embodiments, it may be expedient to arrange the device 100 for determining a body area condition with its light source 106 (the display) and the camera 108 at a distance from the body area 102K that allows a sufficient signal strength of the recorded images. For example, a expedient distance may be less than about 20 cm, for example, between about 1 cm and about 10 cm.

In various embodiments, the display 106 and the front camera 108 of the smartphone 100 may face the body area 102K.

The portable data processing device 100 includes a processor and a memory in various embodiments.

The portable data processing device 100 may be configured in various embodiments to determine at least one body area condition parameter based on the plurality of recorded images.

Various embodiments may use software for determining the at least one body area condition parameter, for example, an app, which may be installed on the portable data processing device 100.

In various embodiments, the at least one body area condition parameter may be determined as a function of time (for example, several times daily, daily, weekly, or with any other temporal dependency) and/or under various environmental conditions (for example, in a high or low humidity, in cold or heat, etc.).

In various embodiments, when the light 112 that has interacted with the body area 102K is recorded, a spectral characteristic of the light 110 radiated from the light source 106 to the body area 102K may be known (also referred to as a known spectral characteristic) and used as a reference. The spectral characteristic can, in various embodiments, have intensities of the light 110 radiated in the different colors, for example, absolute and/or relative intensities. The spectral characteristic of the radiated light 110 may be determined or may have been determined in various embodiments during or after manufacture of the portable data processing device 100.

In various embodiments, to determine the at least one body area condition parameter, the recorded light 112 may be compared to the known radiated light 110 to determine the spectral characteristic of the body area 102K.

In various embodiments, results of calibration measurements, for example, calibration spectral characteristics, which may be performed in a laboratory, for example, may be stored in the data processing device 100 and/or in the external data processing device 222, for example, as part of the software/app that may be utilized to associate the spectral characteristic with a body area condition parameter and/or a body area condition.

In various embodiments, the calibration measurements includes a variety of different body area condition parameters, body area conditions, and combinations of body area condition parameters or body area conditions. For example, calibration measurements may be for water-rich and oil-rich skin, water-poor and oil-poor skin, water-poor and oil-rich skin (for skin having different skin colors respectively), teeth having different natural colors with and/or without plaque having different degrees of coverage, wherein the plaque may or may not be stained with a suitable color, hairstyles in which the hair has substantially uniform or non-uniform color (for example, for a variety of different colors). In various embodiments, the calibration spectral characteristic which has the least deviation (for example, the smallest mean square deviation or another suitable measure for the deviation) from the measured spectral characteristic may be determined as the suitable/most suitable spectral characteristic.

In various embodiments, a plurality of calibration measurements (calibration spectral characteristics) may be provided for one or more of the combinations of body area condition parameters or body area conditions. Measurements of users 102 may be used for this purpose in various embodiments, for example, in addition to the laboratory measurements.

In various embodiments, the at least one body area condition parameter may be determined alternatively or additionally by employing simulated spectral characteristics. In various embodiments, a simulated spectral characteristic may be generated in which, for example, based on a known spectral characteristic of the radiated light 110, based on at least one predetermined content of at least one substance, the spectral characteristic which would result when the light 110 to the body area 102K had interacted with the given substance content would be calculated. The spectral characteristic determined on the basis of the images recorded during the exposure with the different colors may be compared with the simulated spectral characteristic, for example, a mean square deviation of the two spectral characteristics may be determined. In various embodiments, simulated spectral characteristics may be generated for a plurality of predetermined contents of the substance, and by minimizing the mean square deviation, the content of the substance whose simulated spectral characteristic has the smallest deviation to the determined special characteristic may be set as the determined content of the substance.

In various embodiments, contents of different substances may be examined simultaneously by employing the simulated spectral characteristic. For example, an absorption by oxygen-saturated hemoglobin may be simulated simultaneously with absorption by oxygen-poor hemoglobin in the simulated spectral characteristic. With a plurality of simulated spectral characteristics generated in this way, absolute and relative contents of low-oxygen or oxygen-rich hemoglobin may be changed. The simulated spectral characteristics may be compared with the determined spectral characteristic, and based on the simulated spectral characteristic with the least deviation, both the content of oxygen-poor and oxygen-rich hemoglobin may be determined and an oxygen saturation of the skin determined therefrom.

In various embodiments, information determined by the device 100 may be similar to that of an imaging spectrometer having a relatively low spectral resolution. Thus, in various embodiments it may be possible for the described examinations (determination of the body area condition parameter or of the body area condition) to generate a separate spectral characteristic for each pixel in the plurality of recordings. This makes it possible to examine changes in the spectral characteristic from pixel to pixel, for example, for a (color) homogeneity examination, for example, of the hair, the skin or the teeth.

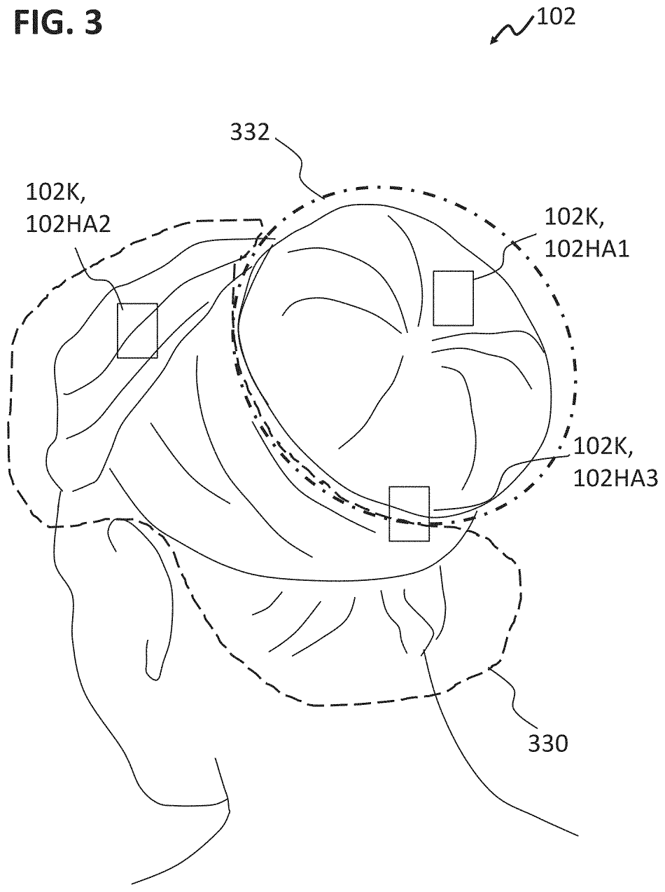

FIG. 3 illustrates such an application example of the method for determining a body condition or a product and/or a care instruction for treating body areas according to various embodiments.

In this case, a hairstyle 330, 332 of a user 102 includes both the user's 102 own hair 330 and foreign hair 332 (artificial and/or natural). Even in a case in which the foreign hairs 332 are selected so that their color looks the same at least when illuminated with a predetermined type of light (for example, daylight), the spectral characteristics may be different, that is, the colors of the hair 330 of the user 102 and the foreign hair 332 may be metamer to each other.

By employing the method for determining the at least one body area condition, the two different colors may be determined on the basis of the determined different spectral characteristics, for example, as described above, for example, in which a body area 102HA1 is directed toward the foreign hair 332 and a second body area 102HA2 towards the user's 102 own hair 330. When arranging the body area, for example, as illustrated for the area 102HA3 in a transition area from the user's own hair 330 to the foreign hair 332, the color difference between the user's own hair 330 and the foreign hair 332 may be determined by a single measurement.

In various embodiments, the determined color difference may be quantified, for example, as a color difference ΔE (also referred to as metamerism index) in the color space used. The color difference in the L*a*b* color space, and even more so in the further developed CIELAB color space, has the property that the calculated color differences ΔE substantially correspond to the respective perceived color difference, that is, colors having a greater color distance in the L*a*b* color space also appear more distinct to a human viewer than colors having a lesser color space, no matter the region of the color space in which the colors are located.

For example, the user 102 may be provided with a recommendation for a (for example, decorative) body treatment product based on determined color differences (color differences ΔE). For example, after examining the user's own hair 330 and several foreign hair products (or otherwise provided color information on the foreign hair products) and determining the corresponding color distances, the hair product having the smallest color difference from the user's own hair 330, ideally a body treatment product having a color difference ΔE<1.0, for example, ΔE<0.5, would be recommended.

In various embodiments, determining colors and/or color differences as described above for a hair example may be applied to other body areas 102K, for example, to skin areas to which a masking cream has been applied, so that a masking cream that has the lowest color difference from a skin color of the user has, is recommended, etc.

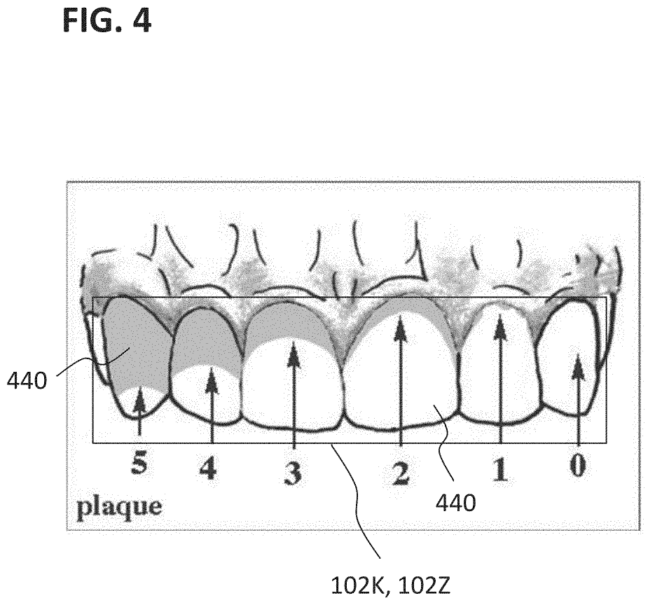

FIG. 4 illustrates a further application example of the method for determining a body condition or product and/or a care instruction for treating body areas according to various embodiments.

According to various embodiments, the body area 102K includes a tooth area 102Z of a user 102.

Teeth 440 of the user 102 may be covered to some degree with coating, particularly plaque, wherein the degrees of coverage typically are quantified with numerical values between 0 (no coverage) and about 5 (about ⅔ or more of the tooth covered). Although the coating, in particular plaque, usually has a color similar to tooth enamel, the coating, in particular plaque, and enamel includes different spectral characteristics, so that a spatial distribution of the coating, in particular of the plaque, similar to that described above for metameric hair colors, and based on which, the degree of coverage of the teeth with coating, in particular the plaque, may be determined as a body area condition. Based on this, a treatment recommendation may be determined, for example, providing guidance for an improved dentifrice technique, suggesting a dental visit, recommending a suitable oral hygiene product such as a suitable toothpaste and/or toothbrush, or the like.

In various embodiments, determining the at least one body area condition, for example, as described above, may be carried out directly by the portable data processing device 100, for example, by the software/app, which may be installed on the portable data processing device 100.

In various embodiments, the at least one determined body area condition parameter may be provided by the portable data processing device, for example, by the software/app. The at least one body area condition parameter can, by way of example, be represented as a value (for example, with arbitrary units), a verbal message, a graphical representation, or communicated similarly, for example, by displaying, for example, on a display 106 of the portable data processing device 100.

In various embodiments, determining the at least one body area condition, for example, as described above, may be carried out indirectly by the portable data processing device 100, for example, by causing the portable data processing device 100 to determine the at least one body area condition by employing an external data processing device 222.

In various embodiments, the images may be provided from the portable data processing device 100, as shown in FIG. 1B, to the external data processing device 222, for example, a central computer or a cloud, which, for example, includes a higher computing power and/or a larger storage capacity than the portable data processing device 100, for example, being transmitted to it, for example, wirelessly as described elsewhere herein, for example. The external data processing device 222 may be configured to determine the at least one body area condition parameter from the images provided, for example, by employing software, for example, an app, for example, as described above.

In various embodiments, the portable data processing device 100 may further be configured to receive and provide the at least one body area condition from the external data processing device 222, for example, as described above. For data exchange with the external data processing device 222, the portable data processing device 100 includes, in various embodiments, a device for the wireless data transmission 224, for example, by employing WLAN, Thread, ZigBee or Bluetooth. In various embodiments, the portable data processing device 100 may be configured to exchange the data with the external data processing device 222 by employing a cable connection.

In various embodiments, the portable data processing device 100 may be configured to determine a body area condition based on the at least one body area condition parameter.

In various embodiments, the portable data processing device 100 may itself be configured, that is, directly, to determine the at least one body area condition, for example, by software, for example, an app, that may be installed on the portable data processing device 100.

In various embodiments, the portable data processing device 100 may be configured to indirectly determine the at least one body area condition, for example, in which the portable data processing device 100 causes the determining of the body area condition by the external data processing device, for example, by employing a software, for example, an app, which may be installed on the external data processing device, for example, similar to that described above for determining the at least one body area condition parameter.

In various embodiments, the software/app for determining the body area condition parameter may be the same as the software/app for determining the body area condition. In various embodiments, for example, in a case where the at least one body area condition parameter is determined by the external data processing device 222, whereas the body area condition is determined by the portable data processing device 100, 100b or vice versa, different software/apps may be used for determining the at least one body area condition parameter and for determining the body area condition.

In various embodiments, determining the body area condition includes comparing the at least one determined body area condition parameter to entries of a database that may have a plurality of body area conditions each having at least one associated body area condition parameter. In various embodiments, more than one body area condition parameter may be associated with one or more of the body area conditions. The association can, for example, have been determined experimentally, for example, in laboratory experiments. Examples of body area conditions may be, for example, dry skin, with at least one assigned value for a water content of the skin (skin moisture) as the body area condition parameter, oily skin, with at least one assigned value for a lipid content, optionally also for a lipid composition of the skin, as the body area condition parameter(s), mature skin, having at least one associated skin collagen content value as the body area condition parameter, optionally further with an associated value for a water content of the skin, a plaque settlement degree of teeth, a tooth color, a hair color, a maximum or average hair color deviation, a skin color, and various other or further body area conditions.

The database may be stored in the portable data processing device 100 and/or in the external data processing device 222 in various embodiments.

In determining the body area condition, the body area condition whose associated body area condition parameter shows the least deviations from the determined body area condition parameter(s) may be determined as the body area condition of the user.

In various embodiments, at least one suitable body treatment product (for example, a body treatment product of the care or decorative cosmetics) and/or at least one care indication may be associated with each of the plurality of body area conditions. The association can, for example, have been determined experimentally, for example, in laboratory experiments.

In various embodiments, literature data may be the basis of assessing suitability of a body treatment product and/or a care instruction in caring for skin, hair or teeth having a given body area condition.

In various embodiments, each of the body area conditions may be associated with a quality value. A body treatment product and/or a care instruction may be rated as suitable for a body area condition when it is to be expected, for example, on the basis of literature data, test results or empirical values, that with a (for example, regular) application of the body treatment product and/or the care instruction, the body area condition of the user is maintained or changed to a body area condition having a higher quality value.

In various embodiments, a body treatment product for a body area condition may be judged suitable when a predetermined maximum difference between a relevant value of the body treatment product value and body area condition is not exceeded. For example, a maximum tolerable value for a color deviation between the hair of the user 102 and a (artificial or natural) hair supplement part may be specified. A hair supplement part having a color having a color deviation from the hair of the user 102 which is smaller than the maximum tolerable value may be judged as suitable and recommended to the user 102, whereas a hair supplement part having a greater color deviation may be judged as unsuitable.

In various embodiments, the hair color of the user 102 may be determined as the body area condition by the device or method for determining the body area condition.

In various embodiments, the color of the hair supplement part may be determined as the body area condition by the device or the method for determining the body area condition, for example, after (for example, probing) arranging the hair supplement part in/on the hair of the user 102, and the color deviation may be calculated by the two determined colors.

In various embodiments, the color of the hair supplement part may be provided as a value determined otherwise or at some other time (for example, by a manufacturer, laboratory, or the like) and may be used as the basis for calculating the color deviation from the hair color of the user 102 determined by the device or the method for determining the body area condition.

In various embodiments, a similar application may be made with other products, which at least, among other things, have a decorative-cosmetic effect, such as dentures (for example, dental implants, crowns, dental repair material), body prostheses (for example, hand or leg prostheses), or the like.

For a denture, for example, a tooth color (for example, at least of adjacent teeth) may be determined by the device for determining the body area condition, and the denture may be colored so as to minimize color deviation between the (natural) tooth color and the denture. For example, for a prosthesis, a skin color (for example, body areas adjacent to the prosthesis) may be determined by the device for determining the body area condition, and the prosthesis may be colored or selected such that a color deviation between the (natural) body areas and the prosthesis is minimized.

In various embodiments, an assessment of a suitability of a body treatment product to improve a body area condition may be confirmed or modified by receiving empirical values of further users 1020 having the same or a similar body area condition, for example, empirical values with regard to treatment success. The empirical values may be provided by the further users, for example, to the external data processing device 222, for example, by employing a wireless data transmission 226. Alternatively, a transmission of the data by a cable may be used. The database in the external data processing device 222 and/or in the portable data processing device 100 may be updated on the basis of empirical values. It is thus possible for the user 102 to always receive an optimal recommendation.

In various embodiments, when determining the body treatment product and/or care recommendations, further information regarding a general health status, age, sex, a further body area condition, eating habits, sports habits, and further behaviors of the user 102 (for example, daily length of stay outdoors/in the sun/in the water, smoking habits, etc.) may be used, for example, by the software/app installed on the portable data processing device 100 and/or on the external data processing device 222. The information can, in various embodiments, be retrieved by the user 102 by the portable data processing device 100, who may enter it into the portable data processing device 100, such as by employing a keyboard, as a voice message, as a selection from a menu presented by the portable data processing device 100, or the like.

For example, when the user 102 provides as additional information that he is spending a lot of time in the water or outdoors, the body treatment product and/or care advisory recommendation may be based on the body area condition of the user 102, for example, the associated body treatment products that, for example, are waterproof and or provide a UV filter are recommended to the user 102.

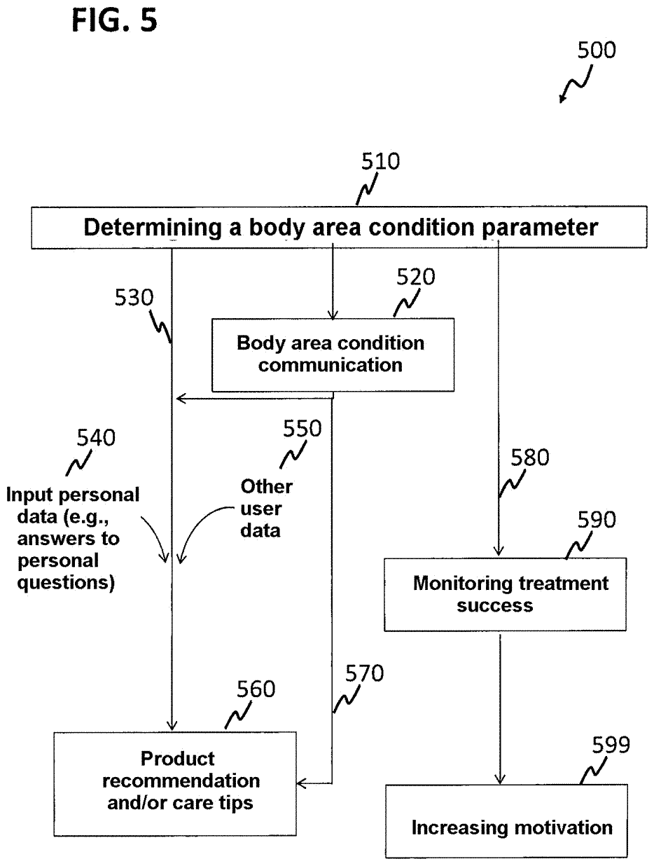

FIG. 5 shows a diagram 500 illustrating a method for determining a product and/or a care instruction for treating a body area according to various embodiments. FIG. 5 may substantially illustrate those processes described elsewhere herein in connection with the method of determining a product and/or a care instruction according to various embodiments.

The method includes, according to various embodiments, the step of determining a body area condition parameter, for example, a measurement of skin moisture, skin elasticity, skin oxygen content, color of the skin, hair and/or teeth, a degree of coverage of teeth with oral plaque, and/or another body area condition parameter, for example, as described above, by the device or method for determining a body area condition (at 510).

In various embodiments, the method further includes the step of determining a body area condition (also referred to as skin type), for example, as described above (at 520).

A body treatment product or care recommendation may be determined (at 560) based on the determined body area condition and/or on the at least one determined body area condition parameter. In this case, in various embodiments (indicated as path 570), only the body area condition determined based on the body area condition parameters may be used.

In various embodiments (identified as path 530), in addition to the at least one body area condition parameter, the personal data as described above may also be consulted (identified with 540) and/or data from other users, for example, their empirical values (identified as 550) may be used.

In various embodiments, furthermore, as illustrated at path 580, a treatment success may be monitored (at 590) by determining the body area condition (at 510). This may be useful, for example, especially during and after a treatment based on the product recommendation (at 560). A treatment success documented by the method for determining the body area condition on the basis of objective values may increase a motivation for the user (at 599).

In various embodiments, as described above, based on the determined body area condition parameters and/or based on the determined body area condition, product recommendations may be derived for body treatment products (for example, care and/or decorative products) which are individually suitable for the user 102 and/or individual care instructions. The product recommendations and/or care instructions may be provided, for example, by employing software, for example, an app.

In various embodiments, the software/app that determines the body area condition parameter and/or the body area condition may be the same as that which determines the product recommendation and/or the care instructions. In various embodiments, different software programs/apps may be used for a part of the various operations or all of the various operations (determining the at least one body area condition parameter, determining a product recommendation, determining a care instruction).

In various embodiments, a treatment success in a treatment, which may have as a goal a positive effect on the determined body area condition parameter or the determined body area condition, may be monitored. In various embodiments, the software/app may enable control and/or tracking of the results by employing a representation (for example, a graphical representation) of the measurement results over time.

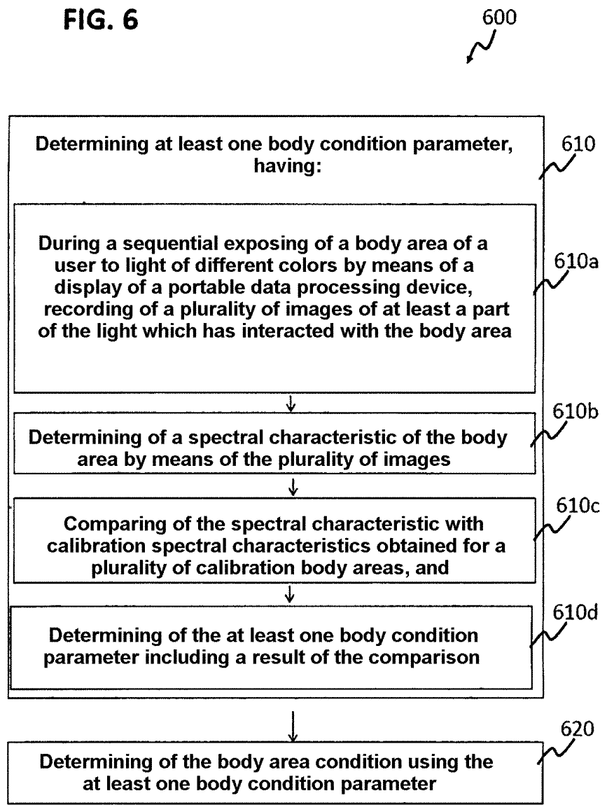

FIG. 6 shows a flowchart 600 of a method for determining a body area condition according to various embodiments.

In various embodiments, a method for determining a body area condition is provided. The method includes determining at least one body area condition parameter (at 610), having: while sequentially exposing a body area of a user to light of different colors by employing a display of a portable data processing device, recording a plurality of images of at least a part of the light which has interacted with the body area (at 610a), determining a spectral characteristic of the body area by the plurality of images (at 610b), comparing the spectral characteristic with calibration spectral characteristics obtained for a plurality of calibration body areas (at 610c), determining the at least one body condition parameter, including a result of the comparison (at 610d), and determining the body area condition using the at least one body area condition parameter (at 620).

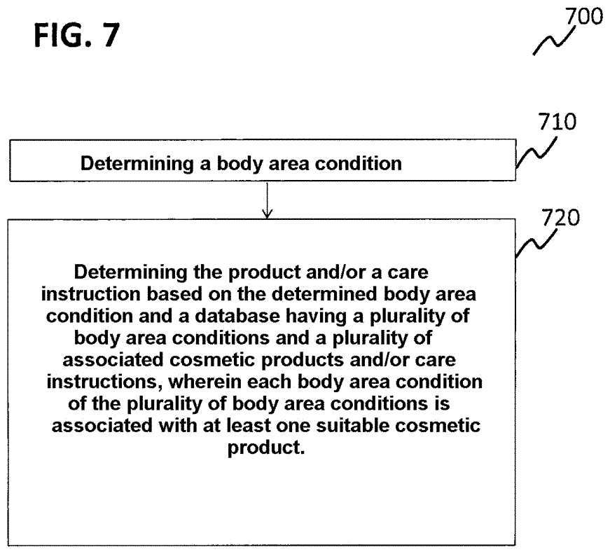

FIG. 7 shows a flowchart 700 of a method for determining a product for skin treatment according to various embodiments.

In various embodiments, a method for determining a product and/or a care instruction for treating a body area is provided. The method includes the steps of: determining a body area condition according to various embodiments (at 710) and determining the product and/or the care instruction based on the determined body area condition and a database having a plurality of body area conditions and a plurality of associated products and/or care instructions, wherein each body area condition of the plurality of body area conditions is associated with at least one suitable body treatment product. (at 720).

Some of the embodiments are described in the context of devices, and some of the embodiments are described in the context of methods. Further advantageous embodiments of the method are apparent from the description of the device and vice versa.

Additionally, while at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for determining a body area condition, the method comprising the steps of:
determining at least one body condition parameter, including:
during a sequential exposing of a body area of a user to light of different colors using a display of a portable data processing device, recording a plurality of images of at least a part of the light that interacts with the body area;
determining a spectral characteristic of the body area from the plurality of images;
comparing the spectral characteristic with calibration spectral characteristics obtained for a plurality of calibration body areas; and
determining the at least one body condition parameter including from a result of the comparison; and
determining the body area condition using the at least one body condition parameter.

2. The method according to claim 1, wherein the step of determining the at least one body area condition parameter includes determining a substance content and/or determining a ratio of substance contents.

3. The method according to claim 1, wherein the at least one body area condition parameter is at least one from a group of body area condition parameters including:
a water content;
a lipid content;
a protein content
an oxygen saturation;
a content of a compound from the group of advanced glycation end products (AGEs);
a content of fine dust;
a pollen content;
a mite content;
a cysteic acid content;
a melanin content;
a silicone content;
a thiol content;
a content of Bunte salt;
a content of disulfide with thioglycolate unit;
a content of antioxidants;
a skin tone;
a tooth color;
a polysaccharide content; and
a degree of coverage of teeth with coating.

4. The method according to claim 1, wherein the at least one body area condition parameter is further defined as a plurality of body area condition parameters.

5. The method according to claim 1, wherein the sequential exposing of the body area of the user to light of different colors includes exposing a plurality of body areas of the user to light of different colors.

6. The method according to claim 4, wherein the step of determining the body area condition includes determining a single body area condition including the plurality of body area condition parameters.

7. The method according to claim 1, wherein the body area has a skin area, a hair area and/or a tooth area.

8. The method according to claim 1, wherein the portable data processing device is a smartphone, a tablet or an iPad.

9. The according to claim 1, further comprising the step of recording at least one dark shot while the body area of the user is not exposed to light of the display of the portable data processing device.

10. A method for determining a product and/or a care instruction for treating a body area, the method comprising the steps of:
determining a body area condition according to the method of claim 1; and
determining a product and/or a care instruction based on the determined body area condition and a database having a plurality of body area conditions and a plurality of associated products and/or care treatments,
wherein each body area condition of the plurality of body area conditions is associated with at least one suitable body treatment product or a suitable care instruction.

11. The method according to claim 10, wherein each body area condition of the plurality of body area conditions is associated with a quality value, and the body treatment product and/or the care instruction is configured for at least one body area condition of the plurality of body area conditions, when based on stored empirical values, an improvement or maintenance of the quality score of the body area condition is expected with the body treatment product and/or the care instruction.

12. The method according to claim 11, further comprising the step of updating the database based on new empirical values from a plurality of users.

13. The method according to claim 1, wherein determining the at least one body area condition parameter and/or determining the body area condition includes the steps of transmitting the plurality of images and/or the result of the comparison to an external data processing device and receiving the body area condition parameter and/or the body area condition.

14. A device for determining a body area condition, the device comprising:
a portable data processing device including:
a display for sequentially exposing a body area of a user to light of different colors; and
a camera for recording a plurality of images of at least a part of the light that has interacted with the body area,
wherein the portable data processing device is configured to determine a spectral characteristic of the body area based on the plurality of recorded images, to determine at least one body area condition parameter based on the spectral characteristic by comparing the spectral characteristic with calibration spectral characteristics obtained for a plurality of calibration body areas, and to determine a body area condition based on the at least one body area condition parameter.

15. The device according to claim 14, wherein the portable data processing device is coupled to an external data processing device by a wireless data transmission, wherein the portable data processing device is configured to determine the at least one body area condition parameter by causing the determination of the at least one body area condition parameter and/or the at least one body area condition; and
wherein the portable data processing device is further configured to receive the at least one determined body area condition parameter and/or the at least one body area condition and to provide the at least one determined body area condition parameter and/or the at least one body area condition to the user.

16. The method according to claim 3, wherein the protein content is a collagen content.

17. The method according to claim 3, wherein the degree of coverage of teeth with the coating is further defined as the degree of coverage of teeth with plaque.

18. The method according to claim 5, wherein the step of determining the body area condition includes determining a single body area condition including the body area condition parameters for the plurality of body areas.
